(19) United States
(12) Patent Application Publication
(10) Pub. No.: US 2014/0174453 A1
(43) Pub. Date: Jun. 26, 2014

(54) LATERAL LIMB SUPPORT DEVICE AND SYSTEM AND METHOD OF USING THE SAME

(71) Applicant: Pro Med Technologies, Inc., Stuart, FL (US)

(72) Inventor: Leon Tonelli Panzica, Stuart, FL (US)

(73) Assignee: Pro Med Technologies, Inc., Stuart, FL (US)

(21) Appl. No.: 14/189,720

(22) Filed: Feb. 25, 2014

Related U.S. Application Data
(63) Continuation-in-part of application No. PCT/US12/70787, filed on Dec. 20, 2012.
Application No. PCT/US12/70787 is a continuation-in-part of application No. PCT/US13/49033, filed on Jul. 2, 2013.
(60) Provisional application No. 61/630,812, filed on Dec. 20, 2011.
(60) Provisional application No. 61/667,282, filed on Jul. 2, 2012.
(60) Provisional application No. 61/961,237, filed on Oct. 9, 2013.
(60) Provisional application No. 61/940,758, filed on Feb. 17, 2014.

(51) Int. Cl. A61F 5/048

(52) U.S. Cl. CPC A61F 5/048
USPC 128/845

(57) ABSTRACT

A lateral limb support system and a method of using the same to facilitate healing of the limb or an appendage thereof are disclosed. The lateral limb support system can include a base; a stanchion, having a first leg and a second leg, wherein the first leg is removably and adjustably coupled to the base; and a limb stabilizing plate removably and adjustably coupled to the second leg. A longitudinal axis of the limb stabilizing plate can be generally perpendicular to the longitudinal axis of the second leg. The system can include both a stationary base and a portable, or wearable, base. This arrangement allows a great deal of flexibility for positioning the wearer's limb in a comfortable position, while still maintaining the proper positioning of the limb to facilitate healing.

### CROSS-REFERENCE TO RELATED APPLICATIONS

This application is a continuation-in-part of International Application No. PCT/US2012/070787, filed Dec. 20, 2012, which claims priority to U.S. Provisional Application No. 61/630,812 filed Dec. 20, 2011, and U.S. Provisional Application No. 61/667,282 filed Jul. 2, 2012; and is a continuation-in-part of International Application No. PCT/US2013/049033, filed Jul. 2, 2013; and claims priority to U.S. Provisional Application No. 61/961,237, filed Oct. 9, 2013, and U.S. Provisional Application No. 61/940,785, filed Feb. 17, 2014.

### FIELD OF THE INVENTION

The application is drawn to devices and systems for supporting limbs, specifically, devices and systems for supporting limbs above a heart of a patient to facilitate healing following an injury or surgical intervention.

### BACKGROUND OF THE INVENTION

Limb supports and braces are known for maintaining body parts in extended positions. Adjustable splints and limb supports can be cumbersome and complicated to use. Foam blocks with holes for supporting an arm are also known for use following hand surgeries. These bulky and uncomfortable devices cause many patients to stop using supports or braces before instructed by their surgeon. Failure to use the supports and braces can result in recurrence of the condition being treated, prolonged healing time, or other complications.

### SUMMARY OF THE INVENTION

A lateral limb support system and a method of using the same to facilitate healing of the limb or an appendage thereof are disclosed. The lateral limb support system can include a base; a stanchion, having a first leg and a second leg, wherein the first leg is removably and adjustably coupled to the base; and a limb stabilizing plate removably and adjustably coupled to the second leg. A longitudinal axis of the limb stabilizing plate can be generally perpendicular to the longitudinal axis of the second leg. The system can include both a stationary base and a portable, or wearable, base. This arrangement allows a great deal of flexibility for positioning the wearer's limb in a comfortable position, while still maintaining the proper positioning of the limb to facilitate healing.

### BRIEF DESCRIPTION OF THE DRAWINGS

FIG. 1 is a side view of a limb support mounted to a stationary base as described herein.

FIG. 2 is an exploded, side view of a limb support mounted to a stationary base as described herein.

FIG. 3 is an exploded, side view of a limb support mounted to a portable base as described herein.

FIG. 4 is a side view of a limb support mounted to a stationary base being used by a user as disclosed herein.

FIG. 5 is a side view of a limb support mounted to a portable base being used by a user as disclosed herein.

FIG. 6 is another front view of a limb support mounted to a portable base being used by a user as disclosed herein.

FIG. 7 is a front view of a limb support mounted to a portable base being used by a user as disclosed herein.

FIG. 8 is a side view of a limb support mounted to a portable base being used by a user as disclosed herein.

As will be understood the drawings accompanying this application are not necessarily drawn to scale.

### DETAILED DESCRIPTION

A limb support and system for controlling the position of a user's limb is disclosed. The support can be anchored by inserting the planar base beneath a mattress, cushion or other object. The support can also be attached to the user via a portable base for portable use of the support. The user's limb can be attached to the limb support in a position that maintains the relevant portion of the limb—for example, a hand after carpal tunnel surgery—in an elevated position, such as a position above the user's heart. It has been determined that this is crucial to facilitate recovery following such surgical procedures. The limb support and system described herein allows the user to maintain the relevant portion of the limb in the proper orientation while sleeping, relaxing or moving from place-to-place when it would otherwise be difficult or impossible to maintain the limb in the preferred position. This results in significantly improved outcomes to the surgical interventions performed on the user/patient.

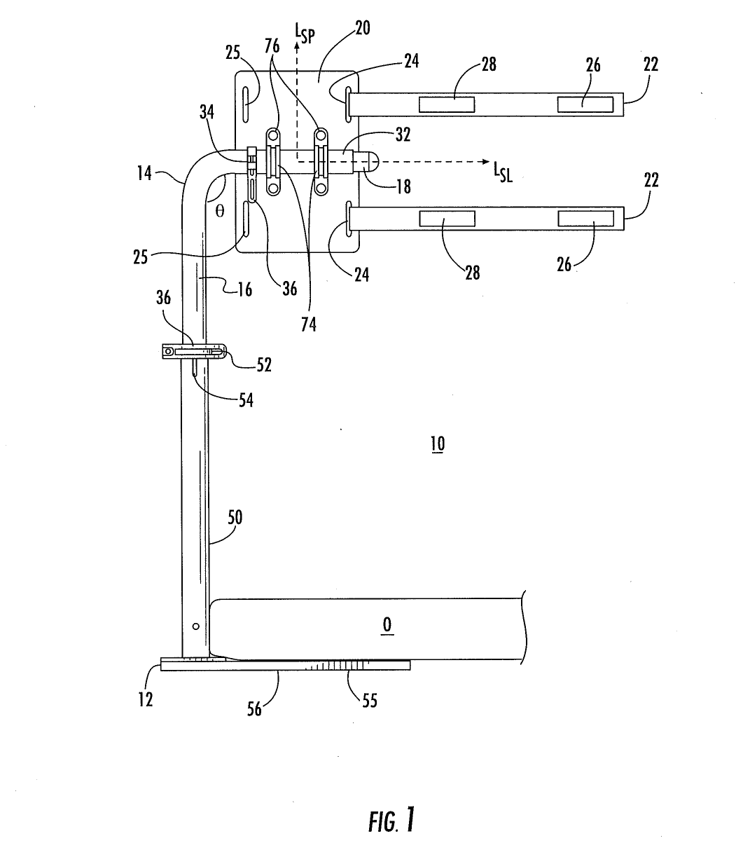

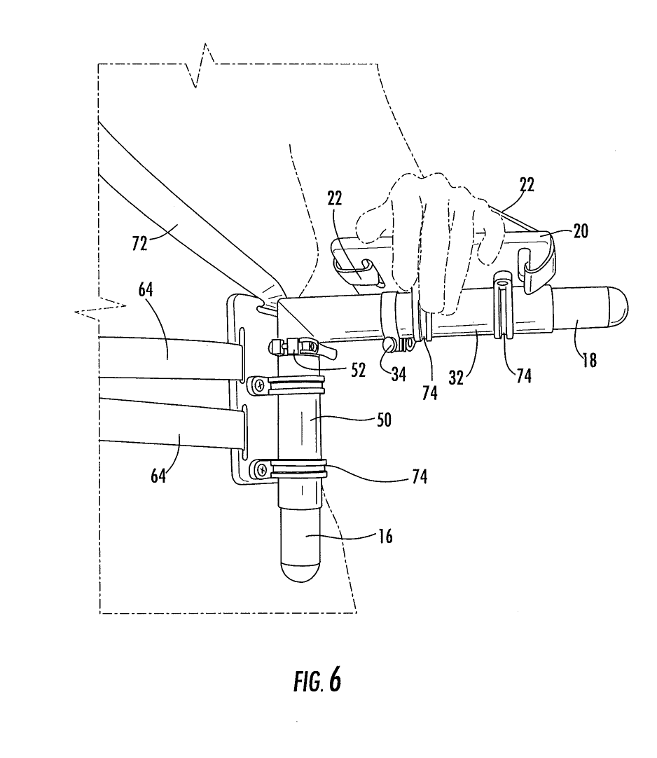

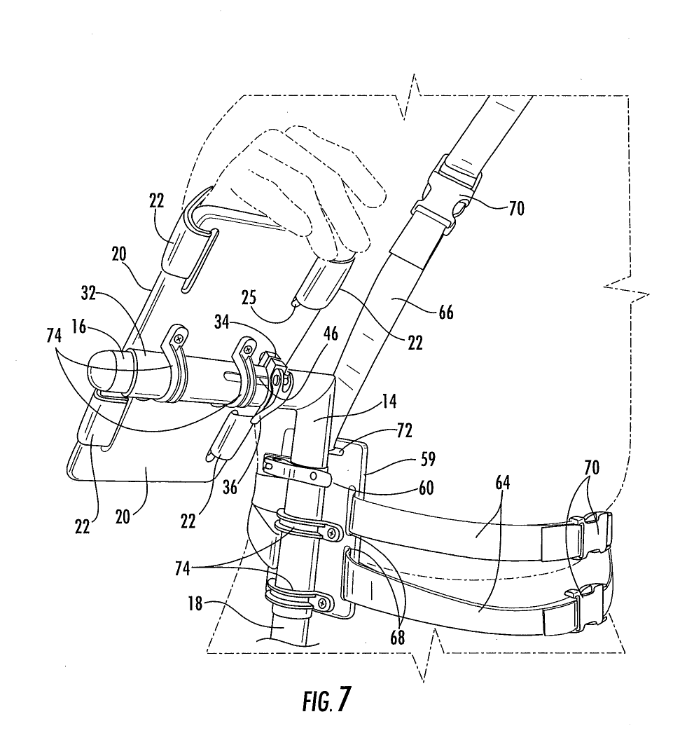

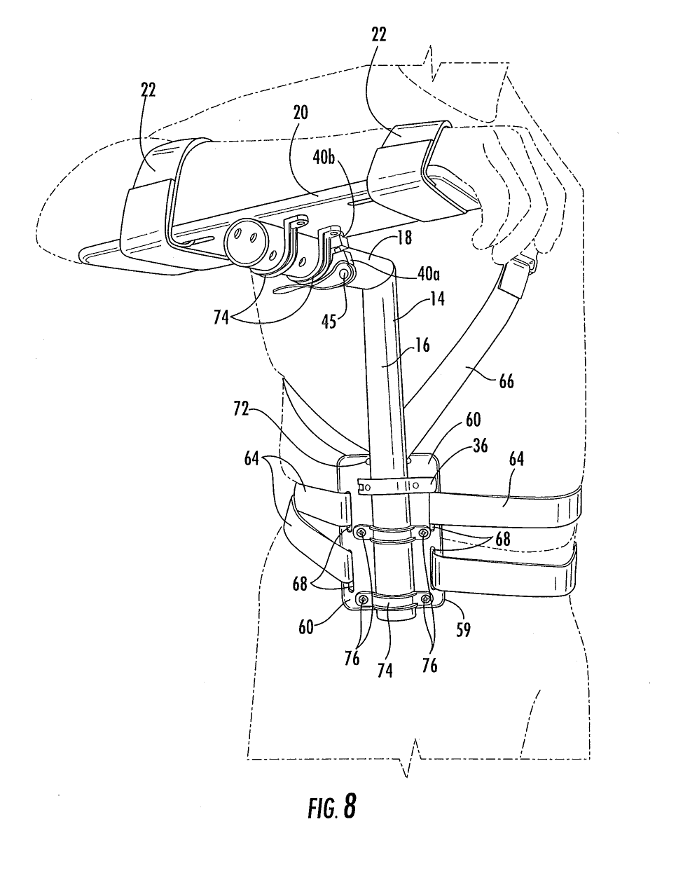

As shown in FIGS. 1-8, the lateral limb support 10 includes a base 12 and a stanchion 14, having a first leg 16 and a second leg 18, where the first leg 16 is removably and adjustably coupled to the base 12. The lateral limb support 10 also includes a limb stabilizing plate 20 removably and adjustably coupled to the second leg 18. The longitudinal axis (Lₛₚ) of the limb stabilizing plate 20 is generally perpendicular to a longitudinal axis (L_{SL}) of the second leg 18.

In some embodiments, such as those shown in FIGS. 1-8, the first leg and the second leg form an angle of approximately 90°. In some embodiments, the first leg 16 is longer than the second leg 18.

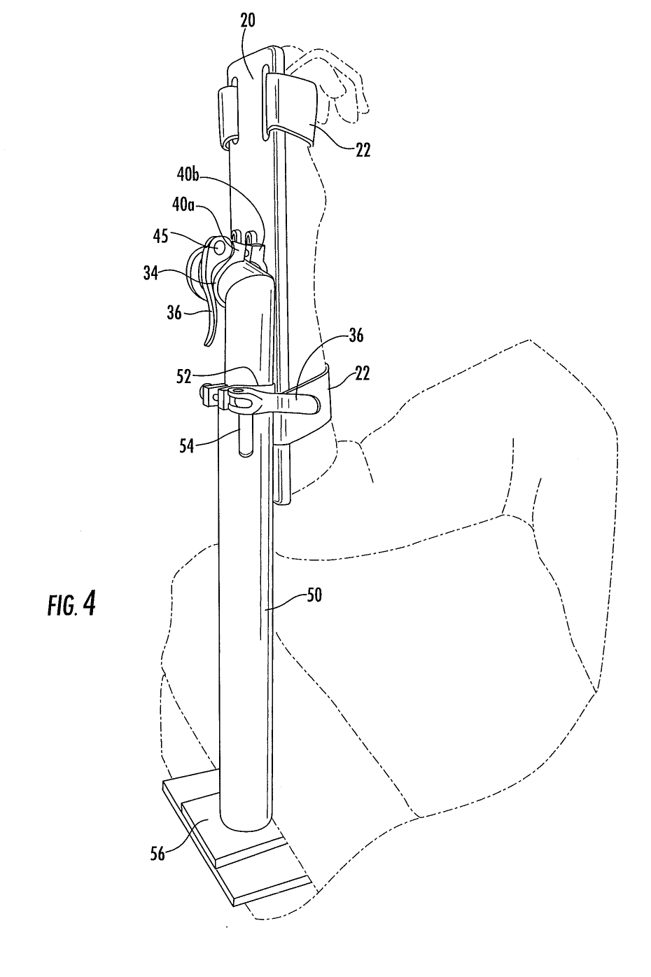

In some embodiments, the lateral limb support 10 can also include one or more straps 22 for securing a limb, such as an arm and/or hand, against the limb stabilizing plate 20. The straps 22 can be coupled to the limb stabilizing plate 20 through respective strap mounting slots 24, which can be positioned proximate a longitudinal edge of the limb stabilizing plate 20. Respective strap receiving slots 25 can be positioned proximate longitudinal edges of the limb stabilizing plate 20 opposite the strap mounting slots 24. As shown in FIG. 1-3, the straps 20 can have a first hook/loop patch 26 proximate a distal end of the strap 22 and a second loop/hook patch 28 extending over an intermediate portion of the strap 22. The distal end of each strap 22 can pass over the limb and then through a strap receiving slot 25, and then fold back on itself so the first hook/loop patch 26 is secured to the second loop/hook patch 28 in a manner that secures the limb to the limb stabilizing plate 20, as shown in FIG. 4-8.

The limb stabilizing plate 20 can be of a sufficient length to support a forearm of a user. For example, the limb stabilizing plate 20 can be approximately as long as an arm from the elbow to the user's palm, so the fingers hang over the edge of the limb stabilizing plate 20.

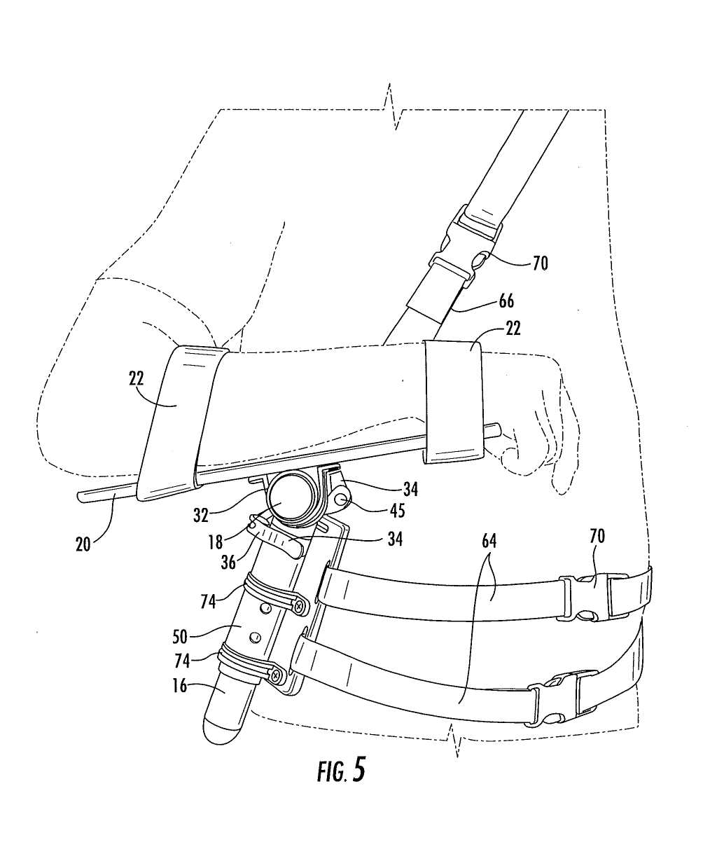

As used herein, “securing a limb” is intended to include securing at least a portion of a limb, which includes the hand or foot, as shown in FIGS. 5, 8-10 and 12. As used herein, “generally” and “approximately” are used to include a deviation of ±15 degrees from the recited direction or configuration, ±10 degrees, or ±5 degrees. Generally is also intended to encompass the direction or geometric property itself. Thus, a structure may include some curvature and be properly described as generally planar and a structure may include a curved transition between the first leg and the second leg and still be described as forming an angle of approximately 90°.

In some embodiments, the limb stabilizing plate 20 is slideably coupled to the second leg 18 and rotatably coupled to the second leg 18. In some more specific embodiments, the second leg 18 comprises a second leg cylindrical portion 30; the limb stabilizing plate 20 is rigidly coupled to a tubular plate mount 32; and the plate mount 32 is adapted to slide over and rotate around the second leg cylindrical portion 30 when they are adjustably coupled together. For example, the tubular plate mount 32 and the second leg cylindrical portion 30 can form a nesting coupling. The limb stabilizing plate 20 can be coupled to the tubular plate mount 32 at approximately the middle of the limb stabilizing plate 20 so that the system can be used interchangeably on both the right side and the left side.

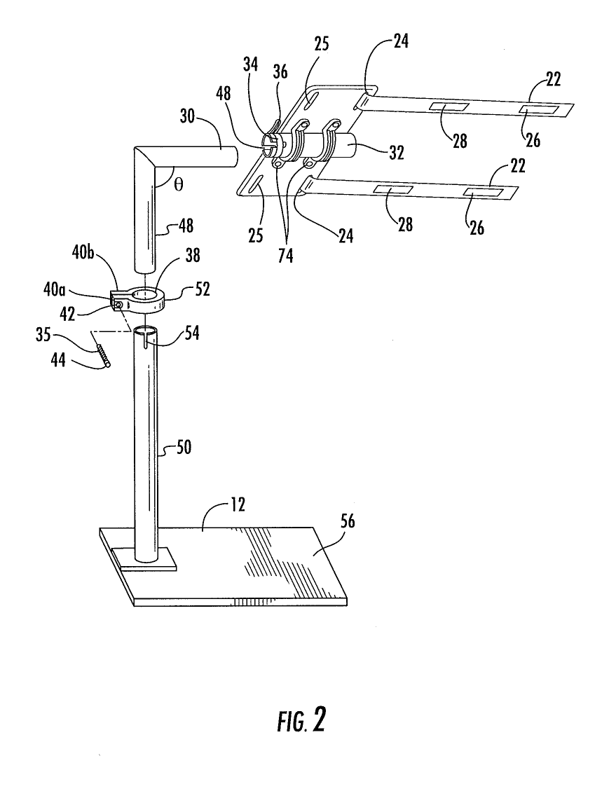

As used herein, “nesting coupling” is used to refer to a coupling where one body fits (or nests) within a cavity of another body. For example, as shown in FIGS. 2 & 3, the plate mount 32 can be a hollow cylinder with an inner diameter slightly larger than an outer diameter of the second leg cylindrical portion 30, which can be hollow or solid. Although not shown, it will be understood that the orientation of nesting couplings can be reversed. The tolerances of the nesting coupling can be tight enough that, in the absence of external forces other than gravity, the two bodies remain coupled without being further secured and diverge 10° or less, or 5° or less, or 2° or less, or 1° or less from the longitudinal axis of the cylindrical components.

In some embodiments, the lateral limb support 10 includes a plate clamp 34 adjustably coupled around the plate mount 32. The plate clamp 34 can be adapted for adjusting between a locked position, as shown in FIG. 4-8, where the plate mount 32 is locked in position relative to the second leg cylindrical portion 30, and an open position, as shown in FIGS. 1 & 3, where the plate mount 32 can slide over and rotate around the second leg cylindrical portion 32. In some embodiments, such as that shown in FIG. 2 with respect to base clamp 52, the plate clamp 34 can be locked and loosened using a set screw 35. In other embodiments, such as those shown in FIGS. 1 & 3, the plate clamp 34 can be locked and opened using a quick release lever 36.

As shown in FIG. 2 with respect to base clamp 52, the plate clamp 34 can include a collar 38 and a securing flange 40a, 40b at each end of the collar 38. In embodiments with a set screw 35, the back securing flange 40b can be threaded for receiving the threaded set screw 35, while the front securing flange 40a can include an orifice 42 that allows the shaft of the set screw to pass, but not the head 44 of the set screw 35.

In quick release embodiments, the quick release lever 36 can be coupled to the back securing flange 40b. In such embodiments, as best shown in FIG. 4, a thickness of the lever base 45 of the quick release lever 36 can be smaller in the open position than in the closed or locked position. As a result, when properly adjusted, the limb stabilizing plate 20 can be locked in position by moving the quick release lever 36 to the closed position, while the limb stabilizing plate 20 can rotate around and slide over the second leg 18 when the quick release lever 36 is in the open position.

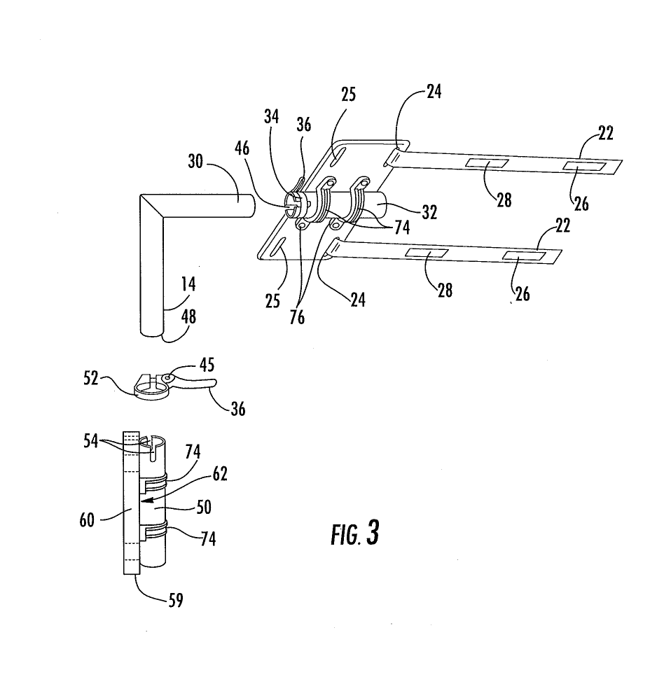

In some embodiments, an end of the plate mount 32 includes at least one plate deformation notch 46 and the plate clamp 34 extends over the at least one plate deformation notch 46. An example of such a configuration is shown in FIGS. 3 & 7. In some embodiments, as shown in FIG. 3, there will be two plate deformation notches 46 on opposite sides of the plate mount 32.

In some embodiments, the first leg 16 includes a first leg cylindrical portion 48, where the base 12 is rigidly coupled to a tubular base mount 50. In such embodiments, the first leg cylindrical portion 48 can be adapted to slide in and rotate within the base mount 50 when they are adjustably coupled together. In some embodiments, the first leg cylindrical portion 48 and the base mount 50 can form a nesting coupling.

In some embodiments, the lateral limb support 10 can also include a base clamp 52 adjustably coupled around the base mount 50. The base clamp 52 can be adapted for adjusting between a locked position, where the base mount 50 is locked in position relative to the first leg cylindrical portion 48, and an open position, where the first leg cylindrical portion 48 can slide through and rotate within the base mount 50.

The base clamp 52 can take any form described with respect to the plate clamp 34. For this reason, the reference numbers used for the components of the base clamp 52 and the plate clamp 34 are identical. Similarly, the above description of the plate clamp 34 components and their orientations applies equally to the components and orientation of the base clamp 52.

In some embodiments, a distal end of the base mount 50 can include at least one base deformation notch 54 and the base clamp 52 extends over the at least one base deformation notch 54. Examples of such a configuration can be seen in FIGS. 1 & 4. As will be understood, the presence of the notches 46, 54 allows the respective plate mount 32 and base mount 50 to deform sufficiently when the clamps 34, 52 are locked that the mounts 32, 50 can clamp down onto the respective cylindrical portions 30, 48.

In some embodiments, such as FIGS. 1-2, the base 12 is a stationary base 55 and the base 12 comprises a base plate 56 and the base mount 50 extends generally perpendicular to a major surface 58 of the base plate 56. In such embodiments, as shown in FIG. 1, the base plate 56 can be positioned under an object (O), such as a mattress or sofa cushion. Thus, the weight of the object (O) and anything resting on the object, such as a person, will stabilize the lateral limb support 10 during use.

In some embodiments, the base 12 is a portable base 59 adapted to be worn by a user, and the base 12 comprises a waist plate 60. As shown in FIG. 3, the base mount 50 can extend generally parallel to a major surface 62 of the waist plate 60. In some embodiments, the lateral limb support 10 can include a belt 64 coupled to the portable base 59, e.g., the waist plate 60, for removably attaching the portable base 59 to a user. In some embodiments, the lateral limb support 10 can also include a shoulder strap 66 coupled to the portable base 59 for removably attaching the portable base 59 to a user.

The belt 64 can be coupled to the waist plate 60 via belt receiving slots 68 positioned proximate opposite lateral sides of the waist plate 60. The belt 64 can include a releasable fastener 70. The belt 64, the fastener 70, or both can used to adjust the length of the belt 64. In some embodiments, as shown in FIGS. 4-8, the portable base 59 can include two belts 64 as described herein.

The shoulder strap 66 can be coupled to the waist plate 60 via a shoulder strap receiving slot 72 located proximate a top edge of the waist plate 60. The shoulder strap 66 can include a releasable fastener 70, as best shown in FIGS. 5 & 7. The shoulder strap 66, the releasable fastener 70, or both, can be used to adjust the length of the shoulder strap 66.

In some embodiments, the plate mount 32 can be coupled to the limb supporting plate 20 by one or more pipe straps 74. The pipe straps 74 can be attached to the plate mount 32 by a screw 76 on each side of the plate mount 32.

In some embodiments, the based mount 50 can be coupled to the waist plate 60 by one or more pipe straps 74. The pipe straps 74 can be attached to the waist plate 60 by a screw 76 on each side of the waist plate 60.

A unique feature of the limb support system 10 is that when a forearm or hand of a user is secured to the limb stabilizing plate 10, the stanchion-support assembly 14, 20 can be moved between the stationary base 55, as shown in FIGS. 1, 2 & 4, and the portable base 59, as shown in FIGS. 3 & 5-8, without the need to unstrap the hand from the limb stabilizing plate 20. For example, user's hand can comfortably and easily be supported by the stationary base 55, as shown in FIG. 4, and then by the portable base 59, as shown in FIGS. 5-8, with a palmar aspect of the hand contacting the limb stabilizing plate 20 at all times. Alternately, the limb can be removed from the limb stabilizing plate 20 when the stanchion-support assembly 12, 20 is moved between the stationary base 55 and the portable base 59.

A method of supporting a limb and/or facilitating healing is also described. The method can include providing a limb support 10 as described herein and attaching a limb of a person to the limb stabilizing plate 20 using the one or more straps 22. The method can also include adjusting the stanchion 14 and the limb stabilizing plate 20 so that the limb stabilizing plate 20 is at or above a level of a heart of a person using the limb support 10. The method can also include the patient using the limb support for a week or more. The method can include attaching the stanchion 14 to the stationary base 55 and a limb to the limb stabilizing plate 20 and attaching the stanchion to the portable base 59 and a limb to the limb stabilizing plate 20, both with the limb stabilizing plate 20 attached and locked to the stanchion 14 as described herein.

The foregoing is provided for purposes of illustrating, explaining, an describing embodiments of the method and system. Modifications and adaptations to these embodiments will be apparent to those skilled in the art and may be made without departing from the scope or spirit of this disclosure. In particular, it is intended that each of the variants described above can be combined with any of the other variants described above.

1. A lateral limb support system, comprising:
a base;
a stanchion, having a first leg and a second leg, wherein the first leg is removably and adjustably coupled to said base; and
a limb stabilizing plate removably and adjustably coupled to the second leg, wherein a longitudinal axis of the limb stabilizing plate is generally perpendicular to a longitudinal axis of the second leg.

2. The lateral limb support system according to claim 1, further comprising one or more straps for securing a limb against said limb stabilizing plate.

3. The limb support system according to claim 1, wherein the first leg and the second leg form an angle of approximately 90°.

4. The limb support system according to claim 1, wherein said limb stabilizing plate is slideably coupled to said second leg and rotatably coupled to said second leg.

5. The limb support system according to claim 1, wherein said second leg comprises a second leg cylindrical portion, wherein said limb stabilizing plate is rigidly coupled to a tubular plate mount, and wherein said plate mount is adapted to slide over and rotate around said second leg cylindrical portion when they are adjustably coupled together.

6. The limb support system according to claim 5, further comprising a plate clamp adjustably coupled around said plate mount, said plate clamp adapted for adjusting between a locked position, where said plate mount is locked in position relative to said second leg cylindrical portion, and an open position, where said plate mount can slide over and rotate around said second leg cylindrical portion.

7. The limb support system according to claim 5, wherein an end of said plate mount comprises at least one plate deformation notch and said plate clamp extends over said at least one plate deformation notch.

8. The limb support system according to claim 1, wherein said first leg comprises a first leg cylindrical portion, wherein the base is rigidly coupled to a tubular base mount, and wherein said first leg cylindrical portion is adapted to slide in and rotate within said base mount when they are adjustably coupled together

9. The limb support system according to claim 8, further comprising a base clamp adjustably coupled around said base mount, said base clamp adapted for adjusting between a locked position, where said base mount is locked in position relative to said first leg cylindrical portion, and an open position, where said first leg cylindrical portion can slide through and rotate within said base mount.

10. The limb support system according to claim 8, wherein an end of said base mount comprises at least one base deformation notch and said base clamp extends over said at least one base deformation notch.

11. The limb support system according to claim 8, wherein said base is a stationary base, wherein said base further comprises a base plate and said base mount extends generally perpendicular to a major surface of said base plate.

12. The limb support system according to claim 8, wherein said base is a portable base adapted to be worn by a user, wherein said base further comprises a waist plate and said base mount extends generally parallel to a major surface of said waist plate.

13. The limb support system according to claim 12, further comprising a belt coupled to said portable base for removably attaching the portable base to a user.

14. The limb support system according to claim 13, further comprising a shoulder strap coupled to said portable base for removably attaching the portable base to a user.

15. The limb support system according to claim 1, wherein said first leg comprises a first leg cylindrical portion, wherein the base is rigidly coupled to a tubular base mount, and wherein said first leg cylindrical portion is adapted to slide in and rotate within said base mount when they are adjustably coupled together; and
wherein said second leg comprises a second leg cylindrical portion, wherein said limb stabilizing plate is rigidly coupled to a tubular plate mount, and wherein said plate mount is adapted to slide over and rotate around said second leg cylindrical portion when they are adjustably coupled together.

16. The limb support system according to claim 15, wherein the first leg and the second leg form an angle of approximately 90°.

17. The limb support system according to claim 16, further comprising a plate clamp adjustably coupled around said plate mount, said plate clamp adapted for adjusting between a locked position, where said plate mount is locked in position relative to said second leg cylindrical portion, and an open position, where said plate mount can slide over and rotate around said second leg cylindrical portion; and
further comprising a base clamp adjustably coupled around said base mount, said base clamp adapted for adjusting between a locked position, where said base mount is locked in position relative to said first leg cylindrical portion, and an open position, where said first leg cylindrical portion can slide through and rotate within said base mount.

18. The limb support system according to claim 17, wherein an end of said plate mount comprises at least one plate deformation notch and said plate clamp extends over said at least one plate deformation notch.

19. A method of supporting a limb, comprising:
providing a limb support system according to claim 1; and
attaching a limb of a person to said limb stabilizing plate using said one or more straps.

20. A method of facilitating healing, comprising:
providing a limb support system according to claim 1; and
attaching a limb of a person to said limb stabilizing plate using said one or more straps.
